(19) United States
(12) Patent Application Publication
(10) Pub. No.: US 2010/0021593 A1
(43) Pub. Date: Jan. 28, 2010

(54) PROCESS FOR MANUFACTURING, BY COLD EXTRUSION, PUFFED INTERMEDIATE FOOD PRODUCTS WHICH ARE STABLE TO HEAT TREATMENT, FROM HYDRATED ANIMAL PROTEINS

(75) Inventors: Nicolas Denis, Paris (FR); Olivier Mercier du Paty de Clam, Guizengeard (FR)

Correspondence Address: SCHWEGMAN, LUNDBERG & WOESSNER, P.A., P.O. BOX 2938, MINNEAPOLIS, MN, 55402, US

(73) Assignee: Ayam SARL, Paris (FR)

(21) Appl. No.: 12/309,121

(22) PCT Filed: Jul. 7, 2006
(86) PCT No.: PCT/FR2006/001631

§ 371 Date: Oct. 2, 2009

(51) Int. Cl. A23P 1/12; G01N 33/02

(52) U.S. Cl. 426/231; 426/516

(57) ABSTRACT

Manufacture of intermediate food products and/or of textured finished products that have been puffed to a greater or lesser extent from hydrated animal proteins without the addition of additives or emulsifiers and/or texturizing agents, which are stable to heat treatment, involving the following succession of steps: (1) a raw initial pulp (A4) is prepared from fish fillets; (2) a series of operations is performed to lead to the production of stabilized pulp (B9) which may or may not be kept in the frozen state; (3) this stabilized pulp is introduced into an evacuated stuffer (110) to be fed into a cold-regulated twin-screw extruder (111) with contra-rotating screws, in which a succession of steps of filling (111A) which may or may not be accompanied by the incorporation of mixing additives (112A/B), of mixing (111B) with a view to homogenizing the ingredients, of shearing (111C) so as to increase the number of potential protein re-attachment sites, of puffing (111 E2with incorporation of air or gas, then of conveying (111F) are carried out in such a way as to obtain a textured food product which has been puffed to a greater or lesser extent and may or may not include inclusions (C11) which is stable to heat treatment and the organoleptic and structural properties of which can be adapted and can differ from those of the raw material used.

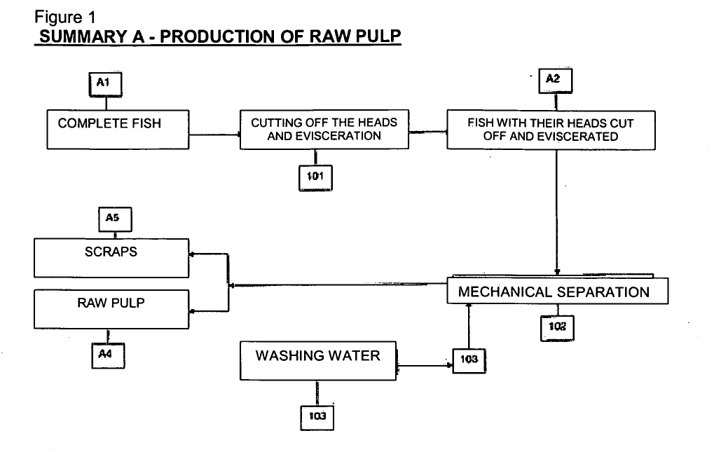

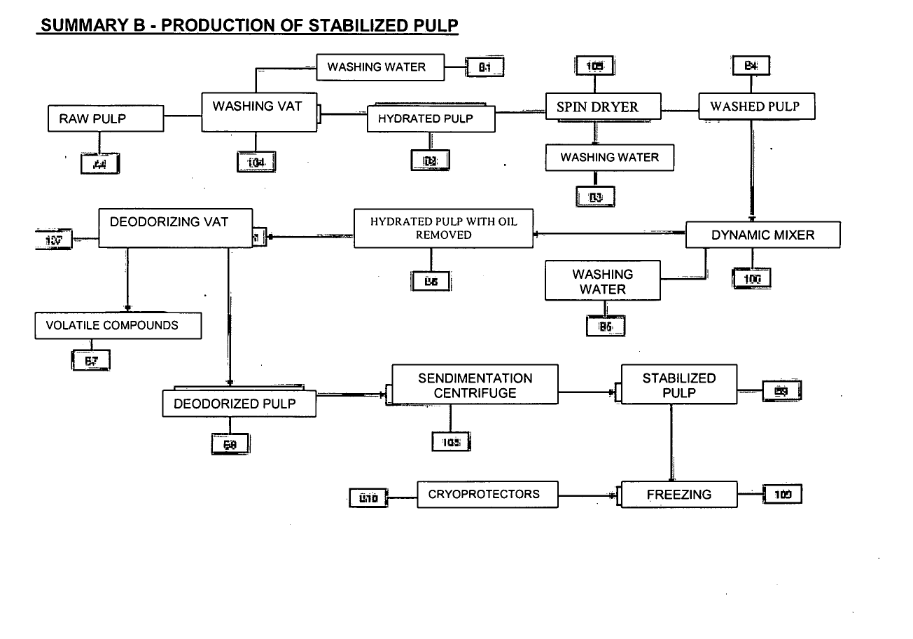

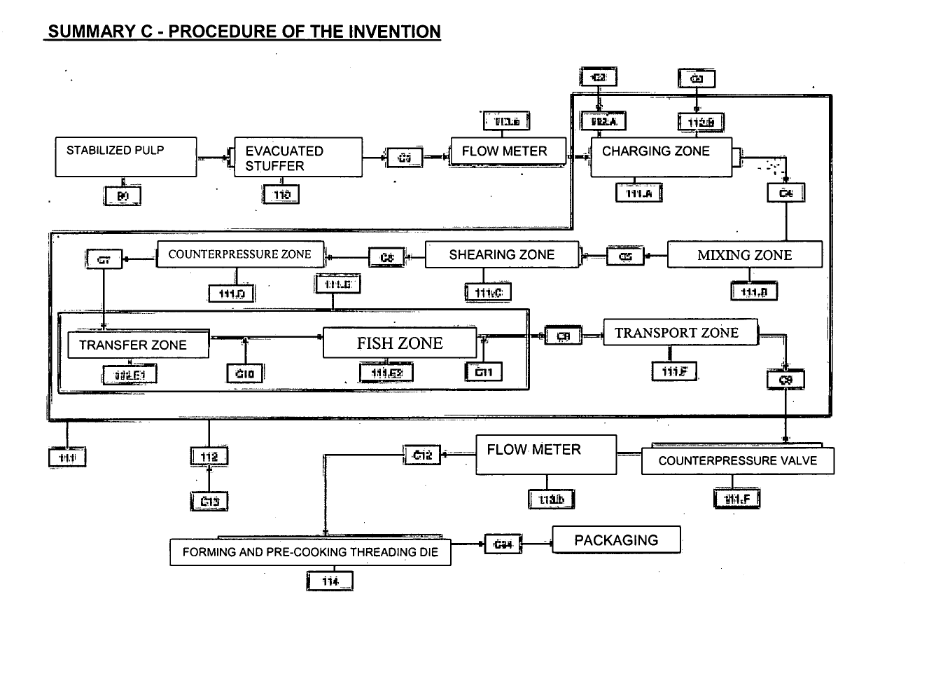

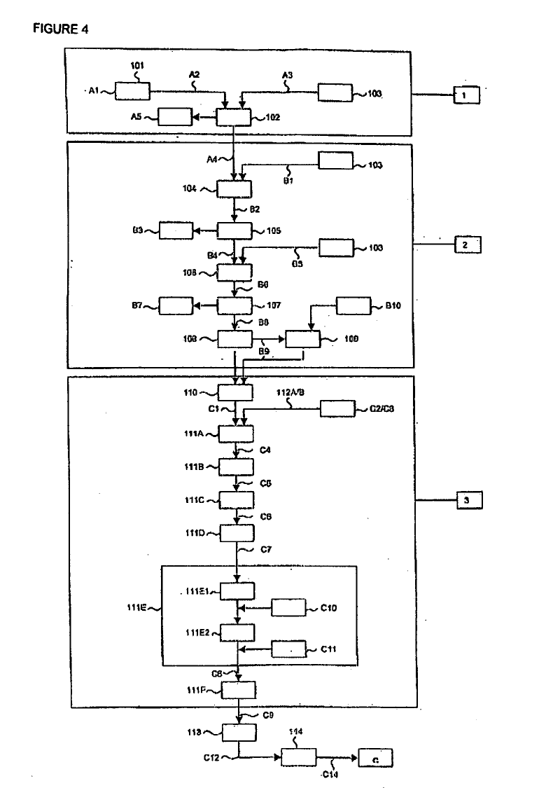

### HISTORY OF THE INVENTION

The invention figures within the food industry and especially in the production of finished textured products or mousses that are puffed to a greater or lesser extent.

The present invention concerns an innovative process for transforming and developing raw material of halieutic origin and more specifically, a production technology for new food products that display a variety of textural and organoleptic qualities, table to heat treatment, made from various marine resources such as fish, molluscs or crustaceans by texturization of these products within a twin screw contra-rotating cold regulated thermo-extruder.

Fish, molluscs or crustaceans have always been widely consumed throughout the world in various forms and their nutritional qualities are largely considered appreciable. Yet, over the last few years, lifestyles and consumer tendencies have become more westernized, giving rise to a certain loss of interest in this type of food when they are presented to consumers in their natural form. For example, the special odor of fish, crustaceans and molluscs, the presence of bones and the inconvenience they may cause are perceived as being unfavorable for consumption of seafood, especially for the younger generations.

Despite this fact, seafood is recognized for its low calorie and high protein content, a reassessment that has furthered stable or even increased the consumption of seafood.

Nevertheless, if these products could be presented in a more easily consumable form, they could be made more attractive for the majority of consumers.

This would thus thwart the tendency to reject fish and encourage more people to consume seafood, numerous attempts have been made to prepare these products in a more attractive and more easily consumable form.

For example, techniques have been developed for producing foods displaying fibrous texture that is comparable to that of fish flesh through light cryogenisation of fish or shellfish pulp, or processes allowing for similar production of crabmeat through modeling and heating of surimi paste in moulds or by extrusion in an agglomerant solution for modifying the chemical characteristics.

Surimi as depicted above is a term used in Japan for characterizing deboned fish meat having undergone a mechanical process prior to being washed with water and mixed with cryoprotectors in such way as to ensure long frozen shelf life.

A large variety of food products have been developed from this hydrated myofibrillary protein concentrate that makes consumption of fish meat protein both possible and pleasant with a taste, flavor and organoleptic aspects that differ from one another.

Nevertheless, when surimi is used as a raw material, hydro soluble protein cannot be used in processing, for it limits the subsequent texturization possibilities. Further, production of such food products requires the implementation of extensive separation, washing and refining operations resulting in substantial losses in yield, which jeopardize the financial viability of production.

Various studies have been conducted in such way as to develop a new food product production process whereby the raw material, such as fish, molluscs or crustacean meat are handled in a relatively simple manner by a mechanism or by a process that provide products intermediate foodproducts with different intrinsic organoleptic qualities that retain the hydro soluble proteins likely to be subsequently texturized and that can be consumed with pleasure.

We have now managed to obtain satisfying results when raw material of halieutic origin such as mechanically deboned fish meat, hereinafter referred to as stabilized pulp, are handled in a twin screw contra-rotating thermo regulated cold extrusion process. These products may then be easily industrially texturized so that they yield a finished product displaying the texture and organoleptic appearance that consumers expect.

### SUMMARY OF THE INVENTION

The present invention proposes a method for transforming, processing and developing a raw material of halieutic origin presented in the form of stabilized pulp, consisting of the utilization of a process of cold extrusion with the help of a twin screw contra-rotating thermo regulated extruder. The said stabilized pulp consists of one or several raw materials of halieutic origin selected among the group of fish, molluscs or crustaceans, or a mixture of the aforementioned raw materials and of a mixture of additives.

The purpose of the process implemented being the production of textured or mousse products, puffed to a greater or lesser extent, from hydrated animal protein, without the addition of additives or texturizing ingredients and/or emulsifiers, stable to heat treatments such as pasteurization, sterilization, associated with one or several vacuum push rods feeding a twin screw contra-rotating extruder upon which one or several regulated air incorporation systems are adapted.

### Context and Principle

In the prior art, traditional mousses or structured food products made from products of halieutic origin are generally produced in two successive steps:
a hot pre-mix with incorporation of ingredients or additives facilitating a pre-emulsion; then
a second step of cold incorporation of proteins so that denaturing by the heat of these latter may be avoided.

These processes of the prior art display a certain number of disadvantages:
they are difficult to implement continuously and are ill adapted to the requirements of an industrial process;
at some 30° C., the temperature required for the first step can be favorable for the proliferation of bacterial flora;
additives (emulsifiers) or technological ingredients (vegetable oil) are required for obtaining a pre-emulsion;
restructuring of fibers, the result of a three-dimensional reorganization of the proteic network, is impossible or antagonistic to the production of a mousse puffed to a greater or lesser extent.

### DETAILED DESCRIPTION OF THE INVENTION

The present invention is described in detail hereafter:

The advantageous implementation of the invention resides, in the first phase, in the possibility of using one or several raw materials of halieutic origin, selected from the group of fish, shellfish or crustaceans, either separately or in a fine mixture.

In one preferred application of the invention, the ground meat used, referred to herein as stabilized pulp, is obtained, in the first phase, by differential mechanical separation according to a gradient of material density, components of raw materials that have been rid of their non-edible parts such as the head and viscera beforehand.

The crush then obtained, primarily composed of sarcoplasmic protein, myofibrillary protein, water and lipids, undergoes a series of processes geared towards eliminating the pro-oxydant compounds naturally contained in the meat of products of halieutic origin, as well as the components likely to interfere with the myofibrillary protein during the final texturization phase by hiding the new sites of protein fixation.

In one preferred application of the invention, the crush obtained by differential mechanical separation is washed, spin-dried, deodorized and then dehydrated in order to obtain a pulp referred to as stabilized pulp.

This stabilized pulp, raw material of the present invention, may or may not be frozen prior to transformation, and cryoprotectors may or may not be added.

It is to be noted that this so-called stabilized pulp bears no correspondence to what is commonly called surimi-base in that the successive separation and purification phases do not lead to obtaining either an exclusively myofibrillary hydrated protein concentrate, nor to obtaining a dry granular product void of hydrophilic properties, but rather to a material having preserved all of its rheological capacities for subsequent re-texturization, despite production yields surpassing 45% based on the whole gross product.

In one preferred application of the invention, the stabilized pulp is introduced into a series of in-line devices including, partially or totally, a vacuum push rod, a twin screw contra-rotating extruder, a back pressure

valve, a die for shaping and pre-cooking and a combined steam/micro-wave cooking oven.

In one preferred application of the invention, the stabilised pulp is introduced into a vacuum push rod that serves to regulate the feeding of a twin screw contra-rotating thermo regulated extruder.

In this configuration, the extruder is composed of a sheath including two contra-rotating screws and equipped at its end with a back pressure valve.

According to a preferred implementation of the invention, the extruder sheath consists of several elements juxtaposed to the interior from which chilled glycolated water is made to circulate in such way as to control the increase in the temperature of the pulp, introduced here, arising from mechanical overheating resulting from the mixing, shearing and compression mechanisms.

Specifically, the extruder will thus have the capacity of carrying out, over a short period, phases such as the transport, compression, mixture, mixing, shearing, heating, puffing and modeling, as per the desired effects upon the material processed.

The factors determining the characteristics of the extruder are essentially linked to the profile of the contra-rotating screws which are comprised of interchangeable elements, each of which have a very specific function.

In one preferred application of the invention, the utilization of a vacuum push rod upstream of the extruder allows for:
ensuring feeding of the extruder with stabilised pulp free from air
regulating the flow of feeding of the extruder and consequently the intensity of the texturization according to the back pressure applied at the outlet of the extruder sheath.

One favored application of the invention implements pressure sensors on each segment of the extruder sheath in such way as to be able to characterize the intensity of the mechanical effects exerted upon the pulp.

In one of the accepted applications of the invention used, the profile of the contra-rotating screws of the extruder shall include, in the following order, or in an order determined in accordance with the desired textural characteristics of the finished product:
a loading area
a pulp mixing area
a shearing area
a back pressure area by inversion of the thread
a destressed area
a transport area

The loading area shall be composed of screw elements bearing a direct and progressive thread ensuring regular feeding and loading of the extruder.

The purpose of the mixing area is to obtain perfect homogenization of the stabilized pulp. The level of this homogenization should be adjusted to the desired appearance of the finished product.

The purpose of the shearing area is to micro-dilacerate the stabilized pulp in such way as to increase the potential number of new sites of protein fixation through formation of a continuous and orderly three-dimensional network.

Shearing intensity shall be controlled, in addition to the value of the entry flow provided by the vacuum push rod and by the length and specific profile of the screw elements, by the back pressure applied on the pulp within the following area comprised of an Archimedes screw bringing about a reflux of the material and a packing plug.

The organization of destructuration sub-zones coupled with adequate chilling (T°<8° C. in the mass) shall uphold the functional properties of the proteins by limiting micro-overheating phenomena occurring in prior art technologies of the curettage type.

The purpose of the destressed area, where the screw elements are of the <<mixer>> type, is to distinguish the preceding area by creating, within the extruder, a depression of which the intensity shall be regulated according to the back pressure applied by a regulating valve located at the axial extremity of the sheath.

The purpose of the transfer area, where the screw elements are of fine pitch, is to facilitate conveyance of the pulp towards the outlet chain by freeing itself of the flow oscillations that can arise in the destressed area due to the functioning principle of disk mixers.

In one preferred application of the invention, the loading area has several material injection inlets, such that they enable the setting of parameters and simultaneous addition of different ingredients intended for modifying the organoleptic aspect of the finished product.

A mixture of additives mentioned above signifies the optional addition of one or several materials or substances to the stabilized pulp in order to adjust its level of humidity, hardness, color, taste or cohesion, thereby procuring for the finished product the desired texture and flavor.

Examples of mixture additives described above include soya-based products and/or wheat-based vegetable proteins such as whole soya, defatted soybeans or their flour, soya protein flour, wheat gluten; yeast; starch such as potato flour or corn flour; cereal flour such as wheat flour and rice flour; milk, powdered milk, casein, egg albumen, fruits and vegetables.

These mixture additives may be used alone or in combination with others.

As described above, the use of mixture additives is facultative. Their matching and mixture relationships are equally variable and adjustable by the operator. When they are employed, they act as an adjuster for moisture or hardness or as a binder for pulps displaying a high moisture content and procure the mouth feel, flavor, elasticity, appropriate flavor and color desired for finished products.

Further, powder or liquid additives such as seasonings, spices, flavor enhancers, thickeners and coloring may be optionally used in the presence or absence of such mixture additives.

The mixture of these additives within the stabilized pulp may be performed beforehand within the mixer, but a preferred application of the invention allows for adjusting the parameters for the incorporation of these ingredients within the extruder loading area.

In one preferred application of the invention, the mixing area consists of a series of flat head screw elements that ensure more or less thorough mixture of the components of the stabilized pulp, to which may be added one or several ingredients. The number of these mixing elements and the induced length of the mixing area are determined according to the operator's wish to have a finished product displaying a more or less homogeneous appearance.

One accepted application of the invention calls for a destressed area made up of two subunits:
a transfer area
a puffing area

The transfer subunit is characterized by fine pitch screw elements that ensure conveyance of the pulp and causing reduced pressure having arisen prior due to the elements of the back pressure area, or even a slight depression enabling the incorporation of gas and/or air.

In one preferred application of the invention, the injection of air and/or gas is carried out using pressure that is greater than the existing pressure in the following puffing subunit and in sufficient quantity for ensuring subsequent product puffing. The quantity of gas injected is controlled and regulated in such way as to obtain finished products that are puffed to a greater or lesser extent.

In the following puffing subunit, the screw elements are of the mixer type ensuring the incorporation of gas and/or air within the pulp reduced to a state of doughy material, as well as a micro-homogenization and product puffing. These two actions are highly dependent upon the pressure within the destressed area, and it is also precision regulated with the help of a back pressure valve located at the axial tip of the extruder sheath.

In another application of the invention, this depression zone is equipped with system of incorporation of solid compounds for the purpose of obtaining inclusions within the proteic matrix constituted by the stabilized pulp, textured and puffed to a greater or lesser extent. These inclusions of size and texture adapted to the operator's wishes shall be introduced at the level of the depression area by a flow control push rod device.

By the mere action of conveyance screws in the subsequent transport area, these inclusions will be thoroughly blended with the textured pulp without dilacerating it.

In the present specific application of the invention, these inclusions shall be comprised of particles of a density greater than that of the textured pulp and may be created from surimi, vegetable or any other food product paste that the operator may be interested in. These inclusions, whether prepared in line or not in parallel with the operations concerned by the present invention, may display variable forms and colors while extracts modifying their taste and flavor in contrast with the characteristics of the proteic matrix coating may have been added.

An advantageous implementation of the invention provides for the utilization, in the extruder sheath outlet, of a back pressure valve with adjustable parameters arranged in such way that the pressure within the destressed area can be precisely regulated.

In connection with the extruder's booster parameters by the vacuum push rod, at the rotation speed of the screws and profile of its components within the destressed area, the restriction of the product is adjusted by this back pressure valve in such way as to (should a difference in flow be recorded between the extruder entry and outlet) allow for an adjustment to be carried out in such a manner as to reinstate equality between the flows and ensure constant and regular functioning within the extruder sheath. In one preferred application of the invention, the product obtained at the extruder outlet, composed of textured stabilized pulp that may or may not contain additives or other ingredients may be immediately heated to undergo heat coagulation.

Conventional heating systems using steam, electricity, gas or similar systems may be used as equipment for heating the product described above. Batch or continuous processing may be employed as long as heat coagulation and pasteurization of the said product may be performed.

In one preferred application of the invention, the heating device is composed of a die inserted into a double sealed enclosure within which steam circulates at an adjustable temperature. This low heating device facilitates progressive coagulation of the proteic matrix without excessive cooking in such way that the flow within the cooking chain may be carried out in a fluid manner.

Advantageous implementation of the invention reflects that this heating chain may, simultaneously with the proteic coagulation, be used as a shaping die in such a way as to provide a finished product with a gauge and form adapted to the operator's wishes.

At this phase of the invention's process, we maintain the possibility of obtaining a textured product displaying a more or less fibrous structure and puffed to a greater or lesser extent, stable to heat treatments of pasteurization and sterilization, with no other additives or ingredients specific to this type of properties.

Thus, in keeping with the present invention, by using stabilized pulp containing one or several raw materials of halieutic origin selected from the group of fish, molluscs or crustaceans according to the operator's financial or commercial interests, or containing a mixture of these raw materials and additives, it is possible to obtain a textured food product, stable to heat treatments, of which the organoleptic features may be adapted and varied from those of the raw material used.

In addition to the innovative aspects exposed above, the interest in this method lies in being able to perform all operations on line and continuously on an industrial scale, without having to process a previous phase of hot pre-mixing for the incorporation of additives or ingredients

Yet another innovative feature of the invention lies in being able to produce a pulp or mousse puffed to a greater or lesser extent without the prior formation of an emulsion.

### DIAGRAM OF THE INVENTION

The different fundamental and essential aspects of the invention shall be taken up again one by one and subsequently clarified, but the invention shall be first well understood in light of the description of the following example, provided as reference to the drawings found in the annexes on which:

figure A is a panel displaying the functional diagram for gross raw material processing leading to the production of gross pulp;

figure B is a panel displaying the functional diagram for gross pulp processing facilitating the production of stabilized pulp;

figure C is a panel displaying the functional diagram for processing of the so-called stabilized pulp for purposes of creating, as per the invention, a finished texturized product, or a mousse more or less puffed and stable to heat treatments.

The process will now be described in reference to figures A, B and C (except where expressly indicated to the contrary) while respecting the chronological order of the operating steps or phases that comprise them.

Although it has been especially studied for processing and developing fish, this process, object of the invention, can be applied to any type of product of halieutic origin, whether molluscs or crustaceans, inasmuch as their meat contains a substantial quantity of myofibrillary protein likely to form a proteic gel following texturization and/or agglomeration by physical or chemical processes.

### Overview A

### Phase 1: Heading and Gutting

While they constitute neither an essential element of the present invention nor the purpose of any of the claims resulting from the present invention, it is specified that whole fish (A1), whether fresh or defrosted, used as a gross basic material, are introduced into a mechanical system (101) that performs effective separation of the head and viscera, or are manually processed, in such was as to conserve only that part which is usually edible (A2).

### Phase 2: Raw Pulp Production

Decapitated, eviscerated fishes (A2) are put into a mechanical separator (102) consisting of a variable-speed endless screw, and then into a cylindrical perforated sieve, allowing a gradual separation of the elements in the introduced matter as the function of a density gradient.

In the course of this differential mechanical separation, wash water (A3) will be injected (103) into the separation tool; the pH and the hardness of the water will be adjusted to solubilize as effectively as possible the pro-oxidant compounds present in the meat as well as the sarcoplasmic proteins responsible for subsequent interactions with the myofibrillar proteins that constitute the matrix of the finished products covered by the present invention.

After this mechanical separation (102), a pulp termed raw (A4) will be recovered on the one hand, and on the other, a dry homogenate of skins and bones (A5).

### Overview B

### Phase 1: Washing the Raw Pulp

Although it is not an essential element of the present invention, nor the object of any of the claims resulting from the present invention, it is noted that the raw pulp (A4) is introduced into a washing tank (104) where said raw pulp is mixed in the presence of wash water (B1) as a follow-up to the solubilizing mechanisms for the undesirable compounds.

### Phase 2: Water Removal

After this washing (104), the hydrated pulp (B2) will be introduced into a centrifuge (105) for the purpose of removing the share of wash water (B3) containing the undesirable solubilized compounds.

### Phase 3: Dynamic Mixing

In order to complete the solubilizing of certain lipidic compounds detrimental to the subsequent operations of protein reattachment, the previously washed pulp (B4) will be introduced into a

dynamic mixer (106) in the presence of wash water (B5) whose pH and hardness will likewise be adjusted. The result will be a defatted, hydrated pulp (B6).

### Phase 4: Deodorizing

This hydrated, defatted pulp (B6) will be introduced into a deodorizing tank (107) where it will be processed, by vacuum extraction, to eliminate the volatile aromatic compounds (B7) responsible for the flavor of fish meat.

### Phase 5: Dehydrating

The hydrated, defatted, and deodorized pulp (B8) obtained will then be introduced into a centrifuge settling tank (108), or possibly into a screw press, to produce a pulp termed stabilized (B9) with a moisture level below 85%.

### Phase 6: Freezing

This stabilized pulp (B9) will then possibly be frozen (109) with or without the addition of cryoprotective agents (B10).

### Overview C

### Phase 1: Feeding the Extruder

The first stage of the procedure covered by the present invention consists of putting the stabilized pulp (B9) into an evacuated stuffer (110).

It is noted that this stabilized pulp (B9), made up of washed, purified, and dehydrated meat from any one of the original halieutic products made up of the group of fishes, mollusks, or crustaceans, will have first been thawed if the operations of production of stabilized pulp and texturation are not carried out continuously.

The evacuated stuffer (110) must have a quantifiable sequential push system, and will consist of two copenetrant Archimedes' screws and an evacuation stage downstream from a supply hopper.

The sequence of pushing and the quantity of product transferred by push sequence can be defined, thereby characterizing the instantaneous flow or the weighted flow coming out of the stuffer, as a function of the regulating instructions for the functioning of the extruder described hereinafter.

The stabilized pump (C1) obtained at the output of the stuffer (110) will contain no air.

### Phase 2: Loading the Extruder

In a preferred application of the invention, the extruder (111) is of the co-rotating twin screw type consisting of an outer cover in which there are two intermeshed screws turning in opposite directions.

The two screws consist of a fluted axis supporting interchangeable elements with various functions.

In one application for the invention, the profile of the screws will contain, in the following order, or in a different order determined by the operator based on the texture characteristics desired in the finished product:
a loading area (111.A)
a mixing area (111.B)
a shearing area (111.C)
a back pressure area (111.D)
an expansion area (111.E)
a transport area (111.F)

According to one preferred implementation of the invention, the outer shell of the extruder (111) will consist of several juxtaposed elements inside which chilled glycolated water (C.13) will be circulated, and of a thermoregulation system (112) for controlling the temperature rises in the pulp introduced therein, caused by the mechanical heating resulting from the mixing, shearing, and compression mechanisms. In one preferred application of the invention the temperature at the center of the injected product must not go above 8° C. in order to avoid a denaturation of the proteins or a caking of the product inside the cover.

Specifically, the extruder (111) will be able to handle, in a short time, such stages as the transport, compression, mixing, kneading, shearing, heating, puffing, and molding, depending on the effects desired on the material treated.

The stabilized pulp (C1) will be moved by the stuffer into the loading area (111.A) of the extruder (111) through an airtight tubing system.

The loading area (111.A) will be made up of screw elements with a uniform and a progressive feed, assuring regular and continuous feed for the extruder.

In one preferred application of the invention, the loading area will consist of several injection ports for the material (112.A; 112.B;) to allow definable and simultaneous additions of different ingredients (C2; C3;) intended to modify the organoleptic aspect of the finished product. A mixture of additives mentioned above signifies the optional addition of several materials or substances to the stabilized pulp in order to adjust its moisture content, its hardness, its color, its taste, or its cohesion, and thus produce in the finished product the desired texture, aroma, and flavor.

Examples of mixing additives described above include products made from soy and/or plant proteins of wheat origin, such as whole soy, defatted soy or its flour, soy protein flour, wheat gluten; yeast; starch such as potato starch or corn starch; cereal flour such as wheat flour or rice flour; milk, powdered milk, casein, egg albumin, vegetables, and fruits.

These mixture additives (C2; C3;) can be used singly or in combinations.

As described above, the use of mixing additives is optional. Their assortment and mix ratios are also variable and adjustable by the operator. When they are used, they play the role of moisture adjuster, hardness adjuster, or binder for the pulps with a high moisture content, and produce the desired mouth feel, the aroma, the elasticity, the flavor, and the color appropriate to the finished products.

In addition, powdered or liquid additives such as seasonings, spices, extenders (flavor enhancers), thickeners, and colorings may be employed at will in the presence or absence of such mixing additives.

The mixing of these additives into the stabilized pulp can be done at the outset in a mixer, but one preferred application of the invention will be the definable incorporation of these ingredients in the loading area (111.A) for the extruder.

In another preferred application for the invention, the loading area will be provided upstream with a flow meter (113.a), making it possible to measure the precise quantity of pulp introduced into the cover of the extruder (111). One specific application involves performing this flow measurement by recording the characteristics of the pushing sequence of the evacuated stuffer (110).

The resulting product (C4) coming out of the loading area (111.A) will be a stabilized pulp containing no air and with mixing additives (C2; C3;) in a heterogeneous manner.

### Phase 3: Mixing the Product

The pulp (C4) will be conveyed by the screws of the extruder (111) toward a mixing area (111.B), the purpose of which will be to complete the homogenization of the pulp and possibly of the pulp with the mixing additives (C2/C3;).

This homogenization may be more or less thorough based on the desired nature of the finished product.

In a preferred application of the invention, the mixing area will be composed of a series of screw elements with a flat profile assuring a more or less thorough mixture of the elements that make up the stabilized pulp, with or without the addition of one or more ingredients. The number of these mixing elements and the resulting length of the mixing area will be determined as a function of the operator's desire to have a finished product with a more or less homogenous nature.

The resulting product is a pulp (C5) containing no air, with or without mix ingredients (C2; C3;) and more or less homogenized.

### Phase 4: Shearing the Product

The pulp (C5) will be conveyed by the screws of the extruder (111) toward a shearing area (111.C), the purpose of which is to micro-shred the stabilized pulp so as to increase the potential number of sites for protein reattachment through the formation of a continuous, orderly, three-dimensional network.

The shearing intensity will be controlled further by the value of the entering flow provided by the evacuated stuffer (110) and by the length and the specific profile of the screw elements, by the back pressure applied to the pulp inside the next area (114.D) made up of screws with a reverse feed causing a reflux of the material and a compaction aperture.

The organization of destructuration sub-zones in combination with adequate cooling (T°<8° C. in the mass) will assure preservation of the functional properties of the proteins by limiting the micro-warming phenomenon that comes up in technologies of the previous cuttering art.

The resulting product (C6) is a pulp containing no air, with a greater or lesser amount of mix ingredients (C2; C3;). and more or less micro-shredded.

### Phase 5: Pressurization of the Product

The pulp (C6) will be conveyed by the screws of the extruder (111) toward a back-pressure area (111.D), the purpose of which will be to create a compacting constriction that creates a pressure increase in the mixture.

This back pressure will be created by the specific profile of the screws, which will have a reverse or an indirect feed.

As a function of the number of elements of the screw at this level and the length of the back pressure area (111.D), the efficiency of the shearing (111.C) can be modulated.

The resulting product (C7) is a pulp containing no air, with a greater or lesser amount of mix ingredients (C2; C3;) and more or less micro-shredded.

### Phase 6: Expansion of the Product

The pulp (C7) will be conveyed by the screws of the extruder (111) toward an expansion area (111.E) of which the purpose will be to individualize the preceding areas and supply the following transfer area (111.F) by creating inside the extruder a vacuum pressure with an intensity regulated as a function of the back pressure applied by a control valve (113) located at the axial end of the cover.

In the application used for the invention, the expansion area (111.E) will be composed of two sub-units:
a transfer area (111.E1)
an expansion area (111.E2)

The transfer sub-unit (111.E1) will be characterized by screw elements with a direct feed assuring conveyance of the pulp (C7) and leading to a drop in the pressure previously created by the elements of the back pressure area (111.D), or even a slight vacuum pressure allowing the incorporation of a gas and/or air (C10).

In a preferred application of the invention, the injection of air and/or gas will be performed at a pressure higher than the existing pressure in the following expansion sub-unit (111.E2) and in sufficient quantity to assure subsequent expansion of the product. The quantity of gas injected (C10) is controlled and regulated in such a way as to produce more or less puffed products.

In the sub-unit following the expansion (111.E2), the screw elements will be of the mixing type to ensure the incorporation of the gas and/or air (C10) into the pulp (C7) reduced to a state of

a pasty matter, as well as a micro-homogenization and an expansion of the product.

These two actions depend greatly on the pressure inside the expansion area (111.E), and the latter will be regulated precisely with a back-pressure valve (113) located at the axial extremity of the extruder cover.

In another application of the invention, this area of vacuum pressure (111.E) will be equipped with a system for incorporating solid compounds (C11) with the purpose of producing inclusions in the protein matrix made up of the stabilized, textured, and more or less puffed pulp.

These inclusions (C11), of a size and texture adapted to the operator's wishes, will be introduced at the vacuum pressure area (111.E2) by a stuffing mechanism with a controlled flow.

Through the mere action of the conveyor screws of the subsequent transport area (111.F), these inclusions will be thoroughly mixed into the textured pulp, without shredding of the latter.

In the present application of the invention, these inclusions (C11) will be made up of particles of a higher density than the textured pulp (C7) and may be made from a paste of surimi, vegetables, or any other food product of interest to the operator. These inclusions, prepared on line or not, in parallel with the operations addressed by the present invention, may be of variable shapes and colors and may or may not have additives of extracts that modify their aroma and flavor in opposition to the characteristics of the embedding protein matrix.

The resulting product (C8) is a more or less puffed pulp, with a greater or lesser amount of mix ingredients (C2; C3 etc) and/or inclusions.

### Phase 7: Product Transfer

The pulp (C8) will be conveyed by the extruder screws (111) toward a transport area (111.F), the purpose of which will be to allow conveying the pulp toward the output nozzle (114) by freeing it from the variations in flow that may occur in the expansion area (111.E) because of the way the mixing disks work.

The elements of the transport area screw (111.F) will be direct feed.

The resulting product (C9) is a more or less puffed pulp with a greater or lesser amount of mix ingredients (C2; C3;) and/or inclusions, and conveyed at a constant flow rate.

### Phase 8: Regulating the Pressure

At the axial end of the extruder sheath (111), a counter-pressure valve is set up, whose purpose is to regulate the pressure within the thrust zone (111.E).

In line with the parameters of the feeding of the extruder by the evacuated stuffer (110), with the speed of rotation of the screws, and with the profile of its constituent elements within the thrust zone (111.E), restricted movement of the product (C9) will be adjusted via this counter-pressure valve (113), so characterized that if a difference in the flow is recorded between the entry to and exit from the extruder, an adjustment may be made in a way to reestablish the equality between the flows and to assure constant and regular work in the interior of the extruder sheath.

A massive flow meter (113.b) will be set up downstream from the counter-pressure valve (113) in order to measure the flow that is leaving in comparison with the flow that is entering, measured upon entry into the extruder by the flow meter (113.a).

The resultant product (C12) is a more or less puffed pulp, with additions of mixture ingredients (C2; C3; etc.) and/or of other matter and sent at a constant output.

### Phase 9: Forming and Pre-Cooking

At the end of the counter-pressure valve (113), a forming and pre-cooking path is set up (114) to allow progressive coagulation of the protein matrix and its simultaneous forming.

Conventional heating systems, using steam, electricity, gas, or similar systems may be used as heating equipment to heat the product described above. Batch treatment or continuous processing may be used as long as the coagulation and the pasteurization of the said product can be accomplished.

In a preferred embodiment of the invention, the heating device is composed of a threading die inserted in a double enclosure within which the steam is made to circulate at a temperature that can be regulated. This gentle heating device allows progressive coagulation of the protein matrix without excess cooking, so that the flow within the cooking threading die can be made fluid.

An advantageous embodiment of the invention would have this heating threading die be able to be used, simultaneously with the protein coagulation, both as a threading die that can produce a finished product section and as a form adapted to the desires of the operator.

The product obtained (C14) will be a texturized pulp, more or less puffed, to which the mixture ingredients (C2, C3, etc.) or other items have been added in an approximately tight manner and/or have been formed.

At this stage of the process of the invention, one will retain the possibility of obtaining a texturized product presenting an approximately fibrous and more or less puffed structure, stable under the thermal treatments of pasteurization and sterilization, without the addition of additives or specific ingredients with such properties.

These finished products (C14) may be packaged in evacuated cartons or in cartons under a modified atmosphere as fresh or semi-fresh products, or canned, with or without the addition of a topping sauce.

As already emphasized, the invention will find a preferred application in the area of the industrial increase in value of fish skin, but it remains applicable generally for the transformation of any type of product of marine origin, whether it be mollusk or crustacean, as long as the skin contains a significant quantity of myofibrous proteins susceptible to being made into a protein gel after texturizing and/or agglomeration by physical or chemical procedures.

It is obvious that the invention is not limited to this example, but that it extends to many variations or equivalents as long as its definition as given in the attached claims is respected.

1.-30. (canceled)

31. A process for texturizing by cold extrusion of puffed food products, stable under thermal treatments, with or without other materials, starting with stabilized pulp made of products of marine origin and including the succession of the following stages taken in order:
a stabilized pulp is prepared (B9);
the stabilized pulp is inserted into an evacuated stuffer (110) that serves to feed a cold-regulated twin-screw extruder (111),
this is followed by a series of operations of mixing, sharing, puffing, and transfer within the extruder;
then the resulting product is inserted into a forming and pre-cooking threading die (114).

32. Process according to claim 31, so characterized in that the stabilized pulp (B9) is a hydrated compound of marine origin proteins in which the production yield, starting from the entire raw product, is greater than 45%.

33. Process according to claim 31, so characterized in that said stabilized pulp (B9) may be frozen, with or without cryoprotectors, before use.

34. Process according to claim 31, so characterized in that the stabilized pulp (B9) is inserted into an evacuated stuffer (110), and has a quantifiable, sequential system of thrust.

35. Process according to claim 31, so characterized in that the evacuated stuffer (110) continuously feeds a twin-screw, contra-rotating extruder (111).

36. Process according to claim 31, so characterized in that the stabilized pulp (B9) may receive additions of mixture attitudes (C2, C3, etc.) in which one may adjust humidity, hardness, color, taste, and cohesion of finished product, and to obtain for the finished product the desired texture, taste, and cohesion of the finished product, and to obtain for the finished product the desired texture, taste, and flavor.

37. Process according to claim 35, so characterized in that the contra-rotating twin-screw extruder is made up of a sheath in which two interlocking screws are turning in opposite directions, and including, in the following order or in a different order, several zones of work with different functions, namely:
a charging zone (111.A)
a mixing zone (111.B)
a shearing zone (111.C)
a counter-pressure zone (111.D)
a thrusting zone (111.E)
a transport zone (111.F).

38. Process according to claim 37, so characterized in that the sheath of the contra-rotating twin-screw extruder (111) is composed of several side-by-side elements on the interior of which a chilled glycol water is circulated in order to control temperature of the pulp, affected by mechanical micro-heating that results from mechanisms of mixing, sharing, and compression.

39. Process according to claim 37, so characterized in that the charging zone (111.A) of the extruder (111) is equipped with screw elements working directly and progressively and from several injection entry areas.

40. Process according to claim 37, so characterized in that the charging zone is supplied upstream with a flow meter (113.a) allowing precise measurement of the quantity of pulp (C1) inserted into the extruder (111).

41. Process according to claim 37, so characterized in that the mixing zone (111.B) is composed of flat profile screw elements that assure an approximately close mixture of elements constituting the stabilized pulp (C1), with or without the addition of one or several mixing ingredients.

42. Process according to claim 37, so characterized in that the shearing zone (111.C) is made up of interlocking gear-type screw elements that assure a more or less forced shredding of the pulp (C5), whose use is to increase the protein re-attachment sites by the formation of a three-dimensional, ordered network.

43. Process according to claim 37, so characterized in that the counter-pressure zone (111.D) is made up of screw elements moving indirectly or in an opposite direction, whose use is to create a compact plug and to increase the pressure within the sheath with the purpose of regulating the shearing effect (111.C).

44. Process according to claim 37, so characterized in that the thrusting zone (111.E) is made up of two sub-units:
a transfer sub-unit (111.E1) and
a puffing sub-unit (111.E2).

45. Process according to claim 44, so characterized in that the transfer sub-unit (111.E1) consists of direct drive screw elements, which ensure the movement of the pulp (C7) that result in a reduction of pressure within the extruder sheath, that is, even a slight depression can allow the incorporation of gas and/or of air (C10).

46. Process according to claim 44, so characterized in that the puffing sub-unit (111.E2) contains mixing type screws that ensure the incorporation of the gas and/or of the air within the pulp (C7), as well as of micro-homogenization and an approximately intense puffing of the product.

47. Process according to claim 37, so characterized in that the product transfer zone (111.F) is made up of direct action screw elements in which the result is to allow the movement of the pulp (C8) toward the exit thread die (114), thus freeing them from the flow oscillations and allowing them to act in the thrusting zone (111.E) based on the principle of the functioning of the mixing discs.

48. Process according to claim 44, so characterized in that at the axial end of the extruder sheath (111), a regulable counter-pressure valve (113) is set up, allowing the regulation of the pressure within the thrust zone (111.E) and of the differential between the flow entering and leaving the extruder.

49. Process according to claim 48, so characterized in that the regulation of the counter-pressure valve is done by the differential measurement of the entering flow, recorded by the mass flow meter (113.a), and the exiting flow, registered by the mass flow meter (113.b).

50. Process according to claim 44, so characterized in that the depression zone (111.E) can be equipped with a system to incorporate solid compounds (C11) with the aim of obtaining the included items within the protein matrix formed by the stabilized, texturized, and approximately puffed pulp (C7).

51. Process according to claim 31, so characterized in that a forming and pre-cooking threading die (114) is set up at the end of a counter-pressure valve (113), thus allowing progressive coagulation of the protein matrix and its simultaneous formation.

52. Process according to claim 51, so characterized in that the forming and pre-cooking threading die (114) made up of a threading die shaped and sectioned according to the needs of the operator, and inserted into a double walled container within which the steam is made to circulate at a regulable temperature.

53. Installation for the implementation of a process according to claim 31, so characterized in that the total of the operations described above are done in an orderly and continuous fashion.

54. Installation for the implementation of a process according to claim 31, so characterized in that the operations described above may be adjusted in a way to obtain a finished product, approximately texturized, approximately fibrous, approximately increased by the ingredients of a mixture, approximately puffed, and approximately increased by inclusions.

55. Process according to claim 31, so characterized in that the finished puffed product that is obtained has thermal stability under the treatments of pasteurization or sterilization that may be implemented later.

56. Process according to claim 31, so characterized in that the puffing that is more or less forced in the product is obtained without additional emulsifiers.

57. Process according to claim 31, so characterized in that the primary material used consists of stabilized pulp, which is made up from one or several products of marine origin, jointly or separately, selected from the group of fish, mollusks, or crustaceans, so long as their skin contains a significant quantity of myofibrile proteins that can be formed into a protein gel after texturizing and/or agglomeration by chemical or physical procedures.

58. Process of texturizing according to claim 31, so characterized in that in addition to the technologies of the state of the art, and particularly with regard to patent WO 03079820, the technology proposed here concerns texturization, starting with stabilized pulp, with full products more or less puffed and stable under thermal treatments, directly consumable, and not with the production of a basic material before ultimately being transformed.

59. Process of texturization according to claim 31, so characterized in that, in contrast to the technologies of the prior art, and particularly with regard to patent EP-A2-0 190 873, the technology proposed here demands imperatively that the temperature within the sheath of the extruder be rigorously below 8° C., and that the end use of this device is to allow the completion of a series of operations leading to the obtaining of a more or less forced puffing.

60. Process of texturization according to claim 31, so characterized in that, contrary to all the claims of the prior art, the proposed technology implements a series of non-linear and adjustable operations of mixing, sharing, transfer, and above all of puffing and of expansion within the extruder, which implies the use of a novel and specific screw profile, such as described in claim 39, and which may result only in a specific adaptation of the standard functions of a twin-screw, contra-rotating cold-regulated extruder, not reproducible based on experience or by chance starting with the elements traditionally used in the processes of the prior art.
